(19) United States
(12) Patent Application Publication
(10) Pub. No.: US 2008/0294097 A1
(43) Pub. Date: Nov. 27, 2008

(54) Portable Infusion Set

(75) Inventors: Jong-Uk Kim, Gyeonggi-do (KR); Sang-Eun Hong, Gyeonggi-do (KR)

Correspondence Address: NIXON & VANDERHYE, PC, 901 NORTH GLEBE ROAD, 11TH FLOOR, ARLINGTON, VA, 22203, US

(73) Assignee: RIBOTKISUL CO., LTD., Asan-si (KR)

(21) Appl. No.: 11/660,287

(22) PCT Filed: Sep. 3, 2005
(86) PCT No.: PCT/KR05/02930

§ 371 Date: May 20, 2008

(30) Foreign Application Priority Data
Sep. 3, 2004 (KR) 10-2004-0070421

(51) Int. Cl. A61M 5/172

(52) U.S. Cl. 604/67

(57) ABSTRACT

Disclosed is a portable infusion set which comprises a supply pump for artificially supplying infusion solution to a tube passage connecting a solution storing vessel and an injection syringe, an elastic tube which is installed at the rear end of the supply pump and which is made of elastic material so that the infusion solution supplied from the supply pump is introduced or discharged through the expansion or contraction of the elastic tube, a supply sensor which detects the expansion or contraction limit of the elastic tube, and a micro-computer which controls power supply to the supply pump according to the signal of the supply sensor. The infusion solution is temporarily stored in elastic tube by the supply pump which is set to on/off by the micro-computer, and then injected to a human body with constant pressure through the contractile force of the elastic tube. By using the portable infusion set of the present invention, infusion solution can be supplied when the storing vessel is below human body and power consumption is lowered by intermittent use of the supply pump making the infusion set light and portable, and overpressure between the supply pump and the injection syringe that occurs when the syringe is clogged is prevented making the use of the infusion set more safe

### TECHNICAL FIELD

The present invention relates to a portable infusion set which supplies infusion solution from a solution storing vessel to an injection syringe through a conventional tube passage. More specifically, the invention relates to a portable infusion set wherein the infusion solution which is discharged from a supply pump for artificially supplying infusion solution is temporarily stored in elastic tube which can be expanded or contracted as needed and then injected to a human body with a constant pressure through the contractile force of the elastic tube.

### BACKGROUND ART

Generally, the infusion set for supplying infusion solution to a human body comprises a tube 180 which is made of silicon and which connects a solution storing vessel 110 and an injection syringe 170 injected to blood vessel, and a controller 160 which is installed at certain points of the passage of the tube 180 and which controls the amount of injected infusion solution, as shown in FIG. 4.

For example, I.V. INFUSION SET of Bukwang Medical Inc. and PROFI I.V. INFUSION SET of Sinchang Medical Inc. and I.V. INFUSION SET of Hyupsung Medical Inc., Korea, use this kind of construction.

Generally, a glass vessel or a compressed pack of flexible synthetic resin is used for the above infusion sets 110. For the controller 160, a roller is connected to the circumference of the tube 180 contacting inclined surface so that the tube moves in the longitudinal direction of the tube. By moving the roller in the longitudinal direction, the pressing position of the tube varies and thereby the amount of infusion solution is controlled through the change in the cross-sectional passage area of the tube 180.

Another method for more precisely controlling the injected infusion solution is disclosed in Korean Utility Model No. 0265972 wherein the method of controlling the size of the hole through which infusion solution passes is used instead of the above method of changing pressing position. Also, DOSI-FLOW series (DOSI-FLOW 10, DOSI-FLOW 20, DOSI-FLOW 30) of LEVENTON, Spain, is used as a controller.

### DISCLOSURE OF INVENTION

### Technical Problem

In the above infusion set as described above, however, the infusion solution storing vessel should be located above human body for the infusion solution to be injected. So the method requires the help of a patron or other fixing means equipped with wheels in order to keep the storing vessel above human body causing many inconvenience.

In other words, since the infusion solution storing vessel should be located above human body for the infusion solution to be injected, the infusion solution storing vessel should be hung at the position above human body by using a hanger member. The hanger member can be installed at certain position of the bed or at the fixing means equipped with wheels for movement.

To solve these problems, an infusion pump which, as shown in FIG. 6, uses electronically operating pump to artificially supply infusion solution has been developed.

The infusion pump as shown in FIG. 6 comprises a tube 280 connecting a solution storing vessel 210 and an injection syringe 270, a supply pump 230 for artificially supplying infusion solution to a tube passage 280, and an air detecting sensor 220 which is installed at the front or rear end of the supply pump 230 in order to detect inclusion of the air in the solution.

In the infusion pump, an electronically operating pump 230 is installed between the solution storing vessel 210 and the injection syringe 270 to artificially supply infusion solution to a tube 280 passage. So, it is not necessary to always maintain the solution storing vessel 210 above human body which is injection point, and convenience for use is enhanced.

As examples of these infusion pumps, infusion pump of Jeong-Sang Techno (Model: CURE-MATE SM-210) and infusion pump of DAIWHA CORPORATION (Model: BIOF 3000) is on the market.

Besides, Korean Patent Publication No. 2003-0014189 discloses a method of supplying infusion solution by pump which uses a rotating force using the magnetic.

In many infusion pumps, in addition to the supply pump 230, additional functions are generally added to the product such as an air detecting sensor 220 for convenience in use and safety. Further, since the amount of the infusion solution injected can be set (for example, 100 ml per hour) by controlling the operating speed of the supply pump 230 or the pressure of the tube 280 through user input, the controller used in conventional infusion pumps is not installed.

Though the infusion pump as described above has the advantage of not affected by the position of the solution storing vessel 230, but it has the disadvantage of continuously operating the supply pump 230 while injection is made since the supply of infusion solution only depends on the pump.

In other words, in case the injection should be made for a long time, the operation time of the conventional infusion pump becomes longer and power consumption is increased lowering energy efficiency. Also, due to the increase in the size of the battery according to the increased power consumption, portability is also lowered.

Of course, most infusion pumps provide the function of using storage-battery equipment to secure the movement of patients. But, as described above, the time of supplying electricity is shortened since the supply pump should operate continuously during injection of infusion solution. Therefore the storage battery needs to be larger in order to increase the operating time while moving making the infusion pump large and inconvenient to carry.

Also, while a patient is sleeping at night, the noise caused by the operation of the pump can disturb the sound sleep of the patient. In addition, the irregular pressure resulting from the operation of the pump cannot provide a constant pressure in the discharge tube 281. Also, when the syringe is clogged, serious safety problem on human body can be caused by the excessive pressure in the discharge tube 281 resulting from the continuous operation of the pump. This will be described clearly later through the pressure measuring graph (FIG. 7).

### Technical Solution

According to the portable infusion set of the present invention, the portable infusion set comprises a supply pump for artificially supplying infusion solution to a tube passage connecting a solution storing vessel and an injection syringe, an elastic tube which is installed at the rear end of the supply pump and which is made of elastic material so that the infusion solution supplied from the supply pump is introduced or discharged through the expansion or contraction of the elastic tube, a supply sensor which detects the expansion or contraction limit of the elastic tube, and a micro-computer which controls power supply to the supply pump according to the signal of the supply sensor.

According to the portable infusion set of the present invention, the micro-computer has the function of stopping the supply pump when expansion limit of the elastic tube is detected by the supply sensor and starting the supply pump when contraction limit of the elastic tube is detected by the supply sensor.

Therefore, infusion solution can be supplied even when the storing vessel is poisoned below human body and since the supply pump operates intermittently, power consumption is lowered making the infusion set light, small and portable,

Also, according to the portable infusion set of the present invention, a controller is installed at certain point of the discharge tube located at the rear end of the elastic tube in order to control the injected amount of the infusion solution. The controller is either an electrically operated controller which is automatically controlled by user input into the automatic infusion set comprising the supply pump, the elastic tube and the supply sensor, or a manually operated controller in which the amount of the infusion solution is manually controlled.

### Advantageous Effects

Therefore, when infusion solution is not injected to the human body under some circumstances, the rise of the pressure in the discharge tube is prevented and by converting the irregular pressure of the supply pump to appropriate normal pressure before injection to the human body the blood pressure is not affected and the infusion set can be used safely.

Also, according to the portable infusion set of the present invention, by including an air detecting sensor which is installed at the tube on the front or rear end of the supply pump in order to detect inclusion of the air, the micro-computer can have the function of stopping the operation of the supply pump when air is detected in infusion solution in the tube by the air detecting sensor.

Therefore the air in the infusion solution is kept from being injected into human body preventing various harmful effects resulting from the injection of air into the blood vessel.

### BRIEF DESCRIPTION OF THE DRAWINGS

FIG. 1 is a block diagram illustrating the construction of the portable infusion set according to the present invention.

FIG. 2 illustrates one embodiment of the portable infusion set according to the present invention.

FIG. 3 is a graph showing the pressure in the discharge tube of FIG. 2.

FIG. 4 is a diagram illustrating the construction of the portable infusion set of prior art.

FIG. 5 is a graph showing the pressure in the discharge tube of FIG. 4.

FIG. 6 is a block diagram illustrating the construction of the portable infusion set of another prior art.

FIG. 7 is a graph showing the pressure in the discharge tube of FIG. 6.

### DESCRIPTION OF THE NUMERAL IN THE DRAWING

10: infusion solution storing vessel 20: air detecting sensor

30: supply pump 40: elastic tube

50: supply sensor 60: controller

70: injection syringe 80: tube

### BEST MODE FOR CARRYING OUT THE INVENTION

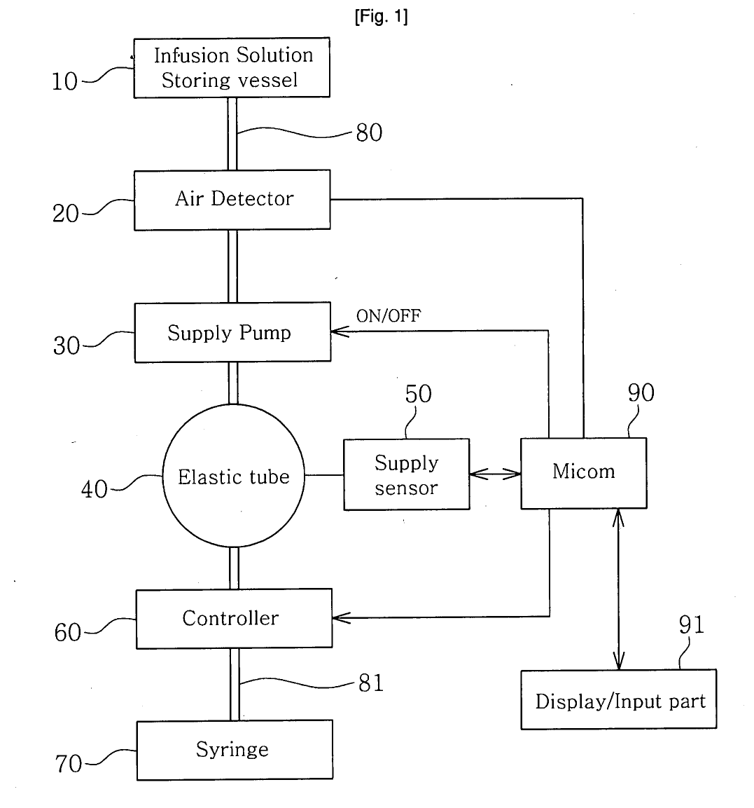

FIG. 1 is a block diagram illustrating the construction of the portable infusion set according to the present invention which shows a series of process regarding the tube 80 passage from the solution storing vessel 10 to the injection syringe 70, and control of the micro-computer.

With reference to FIG. 1, an air detecting sensor 20, a supply pump 30 for artificially moving infusion solution, an elastic tube 40 which temporally stores and discharges infusion solution which is discharged from the supply pump, and a controller 60 to control the amount of supplied infusion solution is installed on the tube 80 passage connecting a solution storing vessel 10 and an injection syringe 70 injected into the human body. Inside the elastic tube 40, a supply sensor 50 which detects the amount of filled infusion solution, a micro-computer 90 which stores predetermined operation that can process the signal from the supply sensor and user input, and display/input part 91 for displaying set value to the user and control the setting is installed.

The air detecting sensor 20 can be installed optionally at the front or rear end of the supply pump 30 in order to detect inclusion of the air in the infusion solution. The air detecting sensor 20, which can be optionally added, is a safety measure that can have the mechanism of automatically stopping the operation of the supply pump 30 when air is detected in infusion solution in the tube by the signal from the air detecting sensor 20.

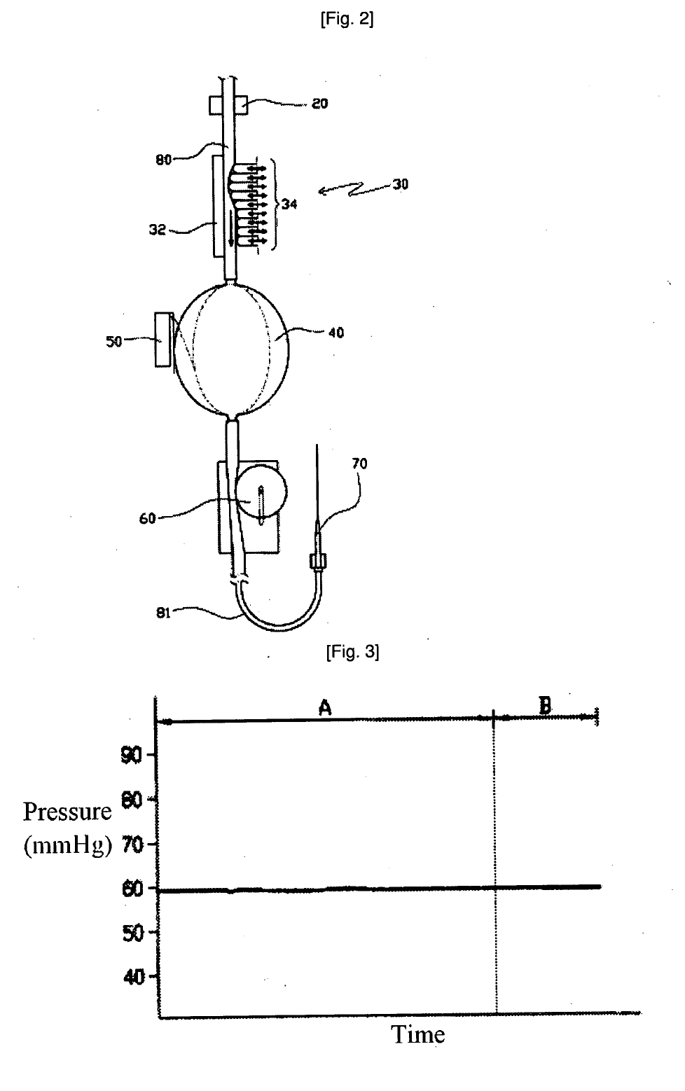

The supply pump 30 moves while pressing the outer surface of the tube 80 to artificially move the infusion solution in the direction of the syringe and described in detail in FIG. 2. Other pumps used generally in the prior art infusion pump can be used.

The infusion solution that flows pressed from the supply pump 30 is introduced into the elastic tube 40. The elastic tube 40 has inlet side into which infusion solution is introduced and outlet side from which infusion solution is discharged. Any material that can elastically expand or shrink like balloon and that is medically verified safe can be used for the elastic tube 40.

The elastic tube 40 acts as a expanding means that temporally stores large amount of infusion solution discharged from the supply pump 30. Also, the elastic tube 40 acts as a contracting means that discharges stored infusion solution to the outlet side with constant pressure and carry out the function of supplying infusion solution to the syringe 70.

On one side of the elastic tube 40 is attached a supply sensor 50 which detects proper expansion or contraction limit of the elastic tube 40. The detected signal of the supply sensor 50 is transferred to the micro-computer 90 and becomes the controlling element of the supply pump 30

When the elastic tube 40 expands while storing the infusion solution discharged from the supply pump 30 and reaches a certain maximum limit, the supply sensor 50 detects the pressure of the elastic tube 40 and transfers signal to the micro-computer 90 and the micro-computer 90 stops the operation of the supply pump 30. Also, when the elastic tube 40 contracts while discharging the infusion solution by elastic force and reaches a certain minimum limit, the supply sensor 50 detects the pressure of the elastic tube 40 and transfers signal to the micro-computer 90 and the micro-computer 90 starts the operation of the supply pump 30 filling the elastic tube 40 with infusion solution. In this way, the supply pump 30 operates intermittently by the control of the micro-computer 90 according to the signal of the supply sensor 50.

Meanwhile, a controller 60 is installed on the discharge tube 81 connecting the elastic tube 40 and the injection syringe 70. The controller 60 can be either a generally used manually-operating controller or an electrically-operating controller which is operated electrically.

The electrically-operating controller can have the type that mechanically presses outer side of the discharge tube 91 or the type that the sectional area of the hole through which the infusion solution passes in automatically controlled. The controller is controlled by the micro-computer 90 according to the user setting on the display/input part.

FIG. 2 illustrates one embodiment of the portable infusion set according to the present invention. With reference to FIG. 2, a detect sensor 20 is provided on the outer side of the tube 80 connected to the infusion solution storing vessel 10. The supply pump 30 contacts on the tube 80 of the elastic panel 32 for predetermined area and a plurality of fingers 34 that can move in right or left direction is provided on the other side of the elastic panel 32.

The fingers 34 repeat projecting and returning operation in a coupled manner in the order from upper to lower finger 34 pressing the tube and moving it the downward direction in the figure. Thus, the infusion solution in the tube 80 moves downward enabling artificial movement of the infusion solution.

This type of supply pump 30 was disclosed in Laid Open Korean Utility Model 1989-0004901 and can be used in the present invention.

The infusion solution discharged through the supply pump 30 is filled into the elastic tube 40 and the elastic tube 40 expands until reaches a certain limit. When the elastic tube 40 reaches a limit, the supply sensor 50 detects the pressure and transfers signal to the micro-computer 90 to cut off the power introduced to the supply pump 30.

Then the infusion solution is discharged to the discharge tube 81 by the contracting force of the elastic tube 40 itself and the elastic tube 40 contracts. When the elastic tube 40 reaches a minimum limit, the supply sensor 50 detects the pressure and transfers signal to the micro-computer 90 to introduce power to the supply pump 30 and the infusion solution is introduced into the elastic tube 40 from the infusion solution storing vessel 10.

In this way, the supply pump 30 needs not to operate continuously but operates intermittently by the signal detected by the supply sensor 50. At this time, since the amount of discharged infusion solution by the supply pump 30 is larger than the amount of discharged infusion solution from the elastic tube 40 by the contracting force of the elastic tube 40, the power of the supply pump 30 can be cut off while infusion solution is discharged from the elastic tube 40 thereby decreasing power consumption substantially. Further, since the filling time is determined by the transfer capability of the supply pump 30, the operating time can be minimized by filling the elastic tube 40 with elastic tube 40 for a short time.

Since, in the present invention, the infusion solution filled in the elastic tube 40 is discharged to discharge tube 81 by pressure of the supply pump, but is discharged after temporarily stored in the elastic tube 40 by contracting force of the elastic tube 40 with a constant pressure keeping the pressure of the discharge tube 81 constant.

Below, the present invention and prior art will be described with reference to the graph which shows the change of pressure against time in the discharge tube.

First, prior art and the present invention will be described in terms of safety.

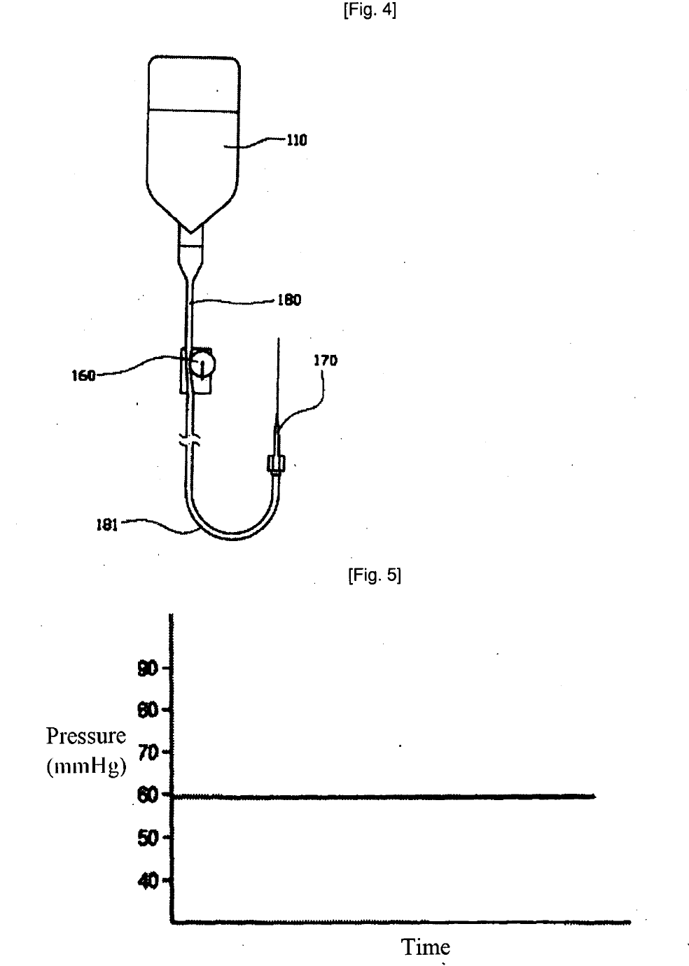

FIG. 5 is a graph showing the change of pressure against time measured by a pressure gauge in the discharge tube 181 of the general infusion set shown in FIG. 4. The figure shows that the pressure in the discharge tube 181 is kept constantly regardless of time. This means that the injection of infusion solution can be carried out under most safe and best circumstance without being affected by the fluctuation of blood pressure of human body such as the change of blood pressure during contraction and relaxation of heart.

In other words, the conventional infusion set is not desirable in that the injecting pressure of the infusion solution is not constant. As described above, this infusion set has disadvantage of having to keep the infusion set above human body and inconvenience in portability.

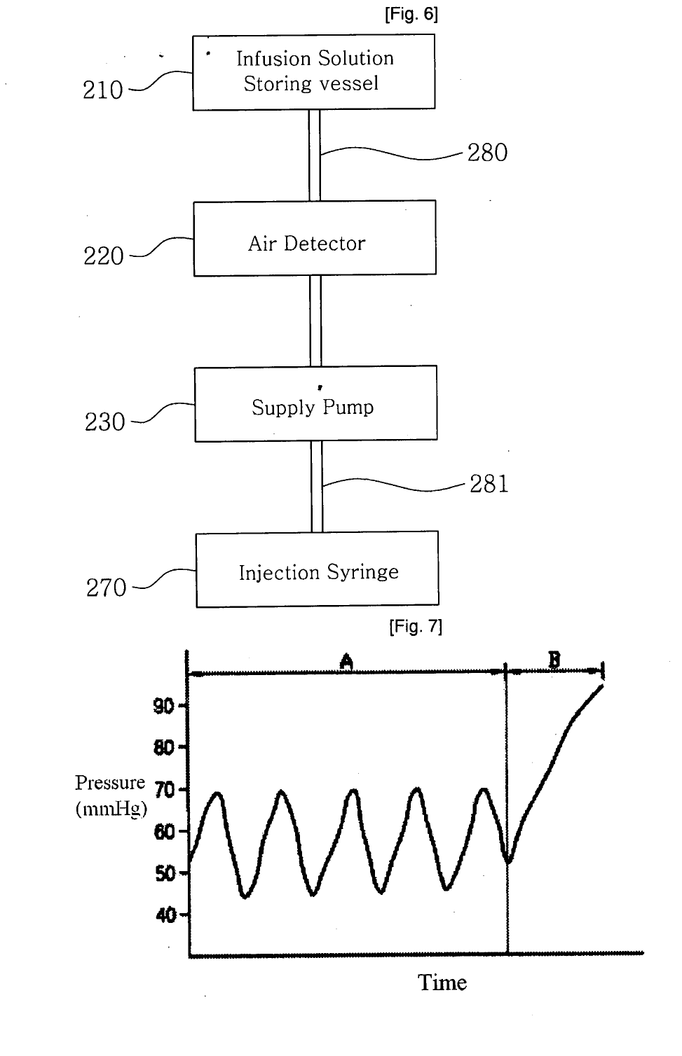

FIG. 7 is a graph showing the change of pressure against time measured by a pressure gauge in the discharge tube 281 of the general infusion set shown in FIG. 6. The experiment is carried out using BIOF 3000 of ‘BIOTRON’. Time interval is not shown since the variation in the period of rise and drop due to the difference in the operating speed of the supply pump depending on the setting of the equipment.

It can be shown, in FIG. 7, that the discharge tube 281 pressure of the infusion pump repeatedly varies by rising and dropping in accordance with the operating pressure of the supply pump.

In other words, in A section where pressure is measured during normal operation of the pump, the graph repeatedly varies by rising and dropping, which means that the discharge strength is not constant depending on the discharge pressure or mechanical operating pressure of the supply pump 230.

Blood pressure while contraction is the blood pressure when blood is discharged into artery by the contraction of heart to supply blood to human body. Normally, the pressure rises to 100 mmHg in artery and drops to 30 mmHg in vein, and the value varies depending on the location of blood vessel. Accordingly, the fluctuation of pressure in the infusion set as in FIG. 7 is not desirable.

Blood pressure of human body varies from many reasons. The pressure can be varied in small or large scale in the human body from the pulsation of heart, breathing, the change in the secretion of hormone, before and after meal or from the environmental reason resulting from the change of posture like lying or sitting.

Hence, if pressure is varied in the discharge tube due to the operation of the supply pump such as infusion pump, the change of pressure of the infusion pump due to the supply pump can affect the change of pressure of the blood vessel into which syringe is injected. When this phenomenon continues for a long time, human body can be harmfully affected in various ways and can cause many undesirable effects.

Meanwhile, in B section in FIG. 7, pressure in the discharge tube 281 is measured while artificially clogging the syringe 270. The syringe 270 can be clogged because of the solidified blood, incorrect injection of the syringe 270 or other reasons, and then the infusion solution is not injected into human body. In this case, the pressure in the discharge tube 281 is seen to rise rapidly for a short time because the supply pump 230 continues to operate supplying infusion solution into the discharge tube 281 although the syringe 270 is clogged. In this case, additional safety equipment should be provided to detect these phenomena.

On the other hand, FIG. 3 is a graph showing the change of pressure against time measured by a pressure gauge in the discharge tube 81 of the infusion set of present invention. In A section where pressure is measured during normal operation of the infusion set, constant pressure is measured without change of pressure. This is the same as the result of measure of the infusion set as shown in FIG. 5.

In other words, the portable infusion set according to the present invention maintains best state without affecting the fluctuation of blood pressure of human body. Also, in B section where the syringe 70 is artificially clogged, the pressure of A section is maintained without change in the pressure. This is because the elastic tube 30 absorbs the pressure change of the supply pump 30 and the infusion solution is injected into the human body by the contracting force of the elastic tube. Hence, there is no chance of affecting human body harmfully even when the syringe 70 is clogged.

The contracting tube of the present invention can be designed to have appropriate pressure according to the material, elasticity and thickness of the tube and can have various contracting force considering the blood pressure of the point of injection.

In addition, the controller 60 can be a commonly used manually-operating controller which is installed at certain point of the discharge tube, or an electrically-operating controller which is automatically controlled by micro-computer 90 according to the user setting of the display/input part 91 in the automatic infusion set comprising the supply pump 30, the elastic tube 40 and the supply sensor 50.

### INDUSTRIAL APPLICABILITY

As described above, according to the portable infusion set of the present invention, infusion solution can be supplied when the storing vessel is below human body which is the injection point. Therefore the portable infusion set is convenient to use when a patient moves on the bed or outside the bed. Also since there is no need for consistently operating the pump, the pump can be operated intermittently and power consumption is lowered enhancing energy efficiency.

Also, according to the portable infusion set of the present invention, a storage battery-type power supply can be used so that miniaturization of the set is possible and the set is convenient to carry.

Also, according to the portable infusion set of the present invention, when infusion solution is not injected to the human body due to clogging of the syringe, rise of the pressure in the discharge tube is prevented and since the variable pressure in the discharge tube due to the supply pump is temporarily stored in the elastic tube and converted to appropriate pressure before injection so as not to affect the blood pressure resulting from the contraction and relaxation of the heart so that the infusion set can be used safely.

1. A portable infusion set comprising:
a supply pump for artificially supplying infusion solution to a tube passage connecting a solution storing vessel and an injection syringe,
an elastic tube which is installed at the rear end of the supply pump and which is made of elastic material so that the infusion solution supplied from the supply pump is introduced or discharged through the expansion or contraction of the elastic tube,
a supply sensor which detects the expansion or contraction limit of the elastic tube, and
a micro-computer which controls power supply to the supply pump according to the signal of the supply sensor.

2. The portable infusion set of claim 1, wherein the micro-computer has the function of stopping the supply pump when expansion limit of the elastic tube is detected by the supply sensor and starting the supply pump when contraction limit of the elastic tube is detected by the supply sensor.

3. The portable infusion set of claim 1, further comprising a controller installed at certain point of the discharge tube located at the rear end of the elastic tube in order to control the injected amount of the infusion solution.

4. The portable infusion set of claim 3, wherein the controller is either an electrically operated controller which is automatically controlled by user input into the automatic infusion set comprising the supply pump, the elastic tube and the supply sensor, or a manually operated controller in which the amount of the infusion solution is manually controlled.

5. The portable infusion set of claim 1, further comprising an air detecting sensor which is installed at the tube on the front or rear end of the supply pump in order to detect inclusion of the air.

6. The portable infusion set of claim 5, wherein the micro-computer includes the function of stopping the operation of the supply pump when air is detected in infusion solution in the tube by the air detecting sensor.
